(19) United States
(12) Patent Application Publication
(10) Pub. No.: US 2026/0301864 A1
(43) Pub. Date: Oct. 1, 2026

(54) GENETIC PREVALENCE ESTIMATOR USING PANEL DISTANCE

(71) Applicant: Laboratory Corporation of America Holdings, Burlington, NC (US)

(72) Inventors: Emil Ragip Keyder, Brooklyn, NY (US); Alix Mary Bénédicte Lacoste, Astoria, NY (US)

(73) Assignee: Laboratory Corporation of America Holdings, Burlington, NC (US)

(21) Appl. No.: 19/490,160

(22) PCT Filed: Jun. 6, 2024
(86) PCT No.: PCT/US2024/032753

§ 371 Date: Dec. 4, 2025

Related U.S. Application Data
(60) Provisional application No. 63/471,575, filed on Jun. 7, 2023.

(51) Int. Cl. G16B 20/20; G16B 20/40

(52) U.S. Cl. CPC G16B 20/20; G16B 20/40

(57) ABSTRACT

This disclosure includes improved methods for analyzing genomic data that include methods of decreasing or removing biases from the genomic data and then analyzing the genomic data from which biases were decreased or removed. For example, estimating the prevalence of a mutations of a specific gene associated with a genetic disorder in a population, determining whether a pair of mutations in a subject are in cis or trans, determining whether a mutation is associated with a genetic disorder, and estimating penetrance of a mutation associated with a genetic disorder.

### RELATED APPLICATIONS

This application claims the benefit under 35 U.S.C. § 119(e) of U.S. provisional application No. 63/471,575, filed Jun. 7, 2023, which is incorporated by reference herein in its entirety.

### BACKGROUND

Estimating prevalence of a genetic disorder due to pathogenic variants of a particular gene is a central problem for drug development, epidemiology, and healthcare. There are a number of datasets of human genome sequencing data, such as gnomAD (https://gnomad.broadinstitute.org/) and UK Biobank (https://www.ukbiobank.ac.uk/), which can be used when estimating prevalence. However, these datasets tend to have various issues that result in biased sampling; and therefore, inaccurate estimates of disease prevalence, such as intentionally excluding subjects with serious disease, in the case of gnomAD, or being heavily biased toward a particular ethnic background, in the case of UK Biobank.

### SUMMARY

Estimating prevalence of genetic disorder in a population using genomic data can guide drug research and development, identify global health concerns, and be used to forecast future healthcare trends. Conventional methods for estimating prevalence of a genetic disorder include estimating using population samples (e.g., using genomic databases) and estimating using models of mutation frequency. These methods typically infer prevalence of a genetic disorder based on the prevalence of a mutation that is associated with the genetic disorder.

Models of mutation frequency are constructed based on different factors, such as the number of nucleotides in a gene, the empirical or estimated mutation frequency by individual codon, etc. Together with a background population allele frequency, and some method of determining whether the resulting alleles are likely to be pathogenic or not pathogenic (i.e., benign), the frequency of de novo disease can be determined. This method can typically only be used for de novo dominant disease, as de novo pathogenic variants in both copies of a given gene are vanishingly rare.

Prevalence of a genetic disorder can also be estimated by determining the prevalence of disorder-associated mutations in population samples (e.g., genomic databases). However, this is challenging because of the frequent sampling biases in genomic databases. For example, gnomAD intentionally excludes subjects with serious disease and UK Biobank is heavily biased towards a particular ethnic background. Databases of gene panel data (i.e., data collected when testing a subject for a genetic disorder using a panel of genes associated with the genetic disorder) also have sampling biases because tested subjects often have or are expected to have the genetic disorder. These sampling biases can skew prevalence estimates. Thus, a central challenge in estimating the prevalence of a genetic disorder in a population is accounting for sampling biases.

The inventors have developed methods for estimating the prevalence of one or more mutations associated with one or more genetic disorders that improve upon conventional methods. Specifically, the disclosed methods reduce or eliminate sampling biases in human genome data prior to estimating the prevalence the one or more mutations in a population. These methods may also be used to estimate prevalence of de novo mutations associated with a dominant disease and one or more mutations associated with a recessive inherited disease. To accomplish this, the disclosed methods may remove a subject from a sample of a population (e.g., database) being analyzed when the subject, prior to genome sequencing, had or was suspected to have the genetic disorder for which prevalence was being estimated.

The disclosed methods take advantage of experimental practices used when testing a subject for a genetic disorder using genome sequencing. A subject having a genetic disorder or suspected of having a genetic disorder may be tested for that disorder using a gene panel. The gene panel comprises a list of genes that when mutated are associated with the genetic disorder or multiple genetic disorders. The genes of the gene panel are referred to as requisitioned genes. When gene panels are sequenced, a superset of genes that comprises the requisitioned genes of the gene panel and non-requisitioned genes (which may be associated with different genetic disorders), are actually sequenced. This superset is referred to as a gene assay. In other words, while there may be many different gene panels for different genetic disorders, there are much fewer gene assays, such that a given gene assay may sequence the genes of many different gene panels. Millions of gene panels have been used to test for many different genetic disorders.

The inventors determined that estimating prevalence of one or more mutations of a specific gene associated with a genetic disorder can be improved by selecting assay data from a subset of subjects who were not tested for the genetic disorder. In other words, prevalence estimates of one or more mutations of a specific gene associated with a genetic disorder may be performed on a subset of subjects whose physician did not order the gene, but whom were sequenced for that gene on an assay. The frequency of one or more disease-causing (aka pathogenic) mutations of a specific gene associated with the genetic disorder in the subset can be used to estimate the prevalence of the genetic disorder in the population. To further decrease bias, the subset may be further reduced by additionally excluding subjects whose gene panel does not include the specific gene, but whose gene panel shares a gene in common with a gene panel that was used to test for the genetic disorder.

Additionally, the inventors determined that gene assay data from the subset of subjects can also be used in other applications besides estimating prevalence. For example, determining whether two mutations in the same gene of a subject are likely to occur in cis or in trans configurations, determining whether a given mutation is associated with a given genetic disorder (i.e. variant interpretation), and estimating the penetrance of a mutation that is associated with a given genetic disorder (e.g., a pathogenic mutation).

The disclosed methods are also more computationally efficient than conventional methods because they involve removing biasing datasets from consideration. Thus, less data may be used to estimate prevalence.

In some embodiments, this disclosure provides a method for estimating prevalence, in a population, of a mutation of a specific gene associated with a genetic disorder, the method comprising: using at least one computer hardware processor to perform: (a) obtaining a plurality of gene assay results for a corresponding plurality of subjects, the plurality of gene assay results comprising (i) gene panel results for a plurality of gene panels corresponding to a respective plurality of sets of requisitioned genes for the plurality of subjects, and (ii) background assay results corresponding to a plurality of non-requisitioned genes; (b) selecting a subset of the plurality of subjects based on the plurality of sets of requisitioned genes and the specific gene associated with the genetic disorder, the selecting comprising, for each candidate subject: determining, based on the plurality of sets of requisitioned genes and the specific gene, a measure of distance between the candidate subject and the specific gene; and selecting the candidate subject to include in the subset of the plurality of subjects when the measure of distance is at least a threshold distance; and (c) estimating prevalence of the mutation using assay results for the selected subset of the subjects.

In some embodiments, determining the measure of distance between the candidate subject and the specific gene comprises: determining a distance between each set of requisitioned genes of the candidate subject and the specific gene to obtain one or more distances; and determining the measure of distance between the candidate subject and the specific gene based on the one or more distances.

In some embodiments, determining the measure of distance between the candidate subject and the specific gene comprises determining a minimum distance of the one or more distances.

In some embodiments, determining a distance between the candidate subject and the specific gene comprises: determining the distance as 0 when the plurality of requisitioned genes of the candidate subject comprises the specific gene; and determining the distance as 1 when (a) the distance is not 0, and (b) the plurality of requisitioned genes of the candidate subject comprises a gene in common with a plurality of requisitioned genes of a second subject.

In some embodiments, determining a distance between the candidate subject and the specific gene comprises:
determining the distance as 2 when (a) the distance is not 0 or 1; and (b) the plurality of requisitioned genes of a second subject, the second subject having a distance 1 from the candidate subject, comprises a gene in common with a plurality of requisitioned genes of a third subject. In some embodiments, the threshold distance is at least 1. In some embodiments, the threshold distance is at least 2.

In some embodiments, estimating prevalence of the mutation comprises estimating prevalence of a mutation associated with a dominant genetic disorder.

In some embodiments, estimating prevalence of the mutation comprises estimating of de novo mutation associated with the genetic disorder.

In some embodiments, estimating prevalence of the mutation comprises estimating prevalence of a mutation associated with a recessive genetic disorder.

In some embodiments, estimating prevalence of the mutation comprises estimating prevalence of an inherited mutation associated with the genetic disorder.

In some embodiments, the plurality of gene panel results comprises at least 100,000 gene panel results.

In some embodiments, estimating prevalence of the mutation comprises estimating using Hardy-Weinberg equilibrium equation.

In some embodiments, estimating prevalence of the mutation comprises estimating in a population of a single sex.

In some embodiments, estimating prevalence of the mutation comprises estimating in a single ethnicity.

In some embodiments, the method further comprises estimating the prevalence of a plurality of mutations of a specific gene that are associated with the genetic disorder, the estimating comprising repeating steps (a)-(c) for each mutation of the plurality of mutations.

In some embodiments, the method further comprises estimating the prevalence of the genetic disorder using the prevalence of the mutation or the plurality of mutations.

In some embodiments, the method further comprises identifying a mutation of the plurality of mutations that has a highest prevalence.

In some embodiments, this disclosure provides a system for estimating prevalence, in a population, of a mutation of a specific gene associated with a genetic disorder, the system comprising: at least one computer hardware processor; and at least one non-transitory computer readable storage medium storing processor executable instructions that, when executed by the at least one computer hardware processor, cause the at least one computer hardware processor to perform: (a) obtaining a plurality of gene assay results for a corresponding plurality of subjects, each gene assay result comprising (i) gene panel results corresponding to a plurality of requisitioned genes and (ii) background results corresponding to a plurality of non-requisitioned genes; (b) selecting a subset of the subjects based on the plurality of requisitioned genes and a specific gene associated with the genetic disorder, the selecting comprising, for each candidate subject: determining a measure of distance between the candidate subject and the specific gene; and selecting the candidate subject to include in the subset of subjects when the measure of distance is at least a threshold distance; and (c) estimating prevalence of the mutation using assay results for the selected subset of the subjects.

In some embodiments, the at least one computer hardware processor stores processor executable instructions that cause the at least one computer hardware processor to perform a method of estimating prevalence described herein.

In some embodiments, this disclosure provides at least one non-transitory computer readable storage medium storing processor executable instructions that, when executed by at least one computer hardware processor, cause the at least one computer hardware processor to perform: a method for estimating prevalence, in a population, of a mutation of a specific gene associated with a genetic disorder, the method comprising: (a) obtaining a plurality of gene assay results for a corresponding plurality of subjects, each gene assay result comprising (i) gene panel results corresponding to a plurality of requisitioned genes and (ii) background results corresponding to a plurality of non-requisitioned genes; (b) selecting a subset of the subjects based on the plurality of requisitioned genes and a specific gene associated with the genetic disorder, the selecting comprising, for each candidate subject: determining a measure of distance between the candidate subject and the specific gene; and selecting the candidate subject to include in the subset of subjects when the measure of distance is at least a threshold distance; and (c) estimating prevalence of the mutation using assay results for the selected subset of the subjects.

In some embodiments, the at least one non-transitory computer readable storage medium storing processor executable instructions, wherein the at least one computer hardware processor stores processor executable instructions that cause the at least one computer hardware processor to perform a method described herein.

In some embodiments, this disclosure provides a method of determining whether two mutations of a specific gene are likely to occur in cis or trans in a subject having or suspected of having a genetic disorder associated with mutation of the specific gene, the method comprising: using at least one computer hardware processor to perform: (a) obtaining a plurality of gene assay results for a corresponding plurality of subjects, each gene assay result comprising (i) gene panel results corresponding to a plurality of requisitioned genes and (ii) background results corresponding to a plurality of non-requisitioned genes; (b) selecting a subset of the subjects based on the plurality of requisitioned genes and the specific gene, the selecting comprising, for each candidate subject: determining a measure of distance between the candidate subject and the specific gene; and selecting the candidate subject to include in the subset of subjects when the measure of distance is at least a threshold distance; (c) identifying the two mutations of the specific gene in the gene assay results of the selected subset of subjects; and (d) determining, using the two mutations identified in (c), whether the two mutations of a specific gene are likely to occur in cis or trans in the subject.

In some embodiments, determining whether two mutations of a specific gene are likely to occur in cis or trans in the subject comprises: (i) determining, using the two mutations identified in (c), that the two mutations are likely to occur in cis in the subject when the two mutations are found in the same gene assay result of the gene assay results at least a threshold number of times; or (ii) determining, using the two mutations identified in (c), that the two mutations are likely to occur in trans in the subject when the two mutations are not found in the same gene assay result of the gene assay results at least a threshold number of times.

In some embodiments, this disclosure provides a method of determining whether a mutation of a given gene is associated with a genetic disorder, the method comprising: using at least one computer hardware processor to perform: (a) obtaining a plurality of gene assay results for a corresponding plurality of subjects, each gene assay result comprising (i) gene panel results corresponding to a plurality of requisitioned genes and (ii) background results corresponding to a plurality of non-requisitioned genes; (b) selecting a first subset of the subjects based on the plurality of requisitioned genes and the given gene, the selecting comprising, for each candidate subject: determining a measure of distance between the candidate subject and the given gene; and selecting the candidate subject to include in the first subset of subjects when the measure of distance is at least a threshold distance; (c) selecting a second subset of the subjects, the selecting comprising, for each candidate subject: selecting the candidate subject to include in the second subset of subjects when the candidate subject has, is suspected of having, or is being tested for the genetic disorder; and (d) determining whether the mutation is associated with the genetic disorder using gene assay results of the first subset of subjects and gene assay results of the second subset of subjects.

In some embodiments, determining whether the mutation is associated with the genetic disorder comprises: (i) determining a first frequency of the mutation in the first subset of subjects and a second frequency of the mutation in the second subset of subjects; and (ii) determining that the mutation is associated with the genetic disorder when the second frequency is greater than the first frequency.

In some embodiments, this disclosure provides a method of estimating the penetrance of a pathogenic mutation in a specific gene associated with a genetic disorder, the method comprising: using a computer hardware processor to perform:
(a) obtaining a plurality of gene assay results for a corresponding plurality of subjects, the plurality of gene assay results comprising (i) gene panel results for a plurality of gene panels corresponding to a respective plurality of sets of requisitioned genes for the plurality of subjects, and (ii) background assay results corresponding to a plurality of non-requisitioned genes;

(b) selecting a subset of the plurality of subjects based on the plurality of sets of requisitioned genes and the specific gene associated with the genetic disorder, the selecting comprising, for each candidate subject: determining, based on the plurality of sets of requisitioned genes and the specific gene, a measure of distance between the candidate subject and the specific gene; and selecting the candidate subject to include in the subset of the plurality of subjects when the measure of distance is at least a threshold distance; and (c) estimating the penetrance of the mutation using the gene assay results of the subset of the plurality of subjects.

In some embodiments, estimating penetrance using assay results for the selected subset of the plurality of subjects comprises estimating based on the frequency of the mutation in the subset of subjects.

In some embodiments, this disclosure provides a method for estimating prevalence, in a population, of a genetic disorder associated with one or more mutations of a specific gene, the method comprising: using at least one computer hardware processor to perform: (a) obtaining a plurality of gene assay results for a corresponding plurality of subjects, the plurality of gene assay results comprising (i) gene panel results for a plurality of gene panels corresponding to a respective plurality of sets of requisitioned genes for the plurality of subjects, and (ii) background assay results corresponding to a plurality of non-requisitioned genes; (b) selecting a subset of the plurality of subjects based on the plurality of sets of requisitioned genes and the specific gene associated with the genetic disorder, the selecting comprising, for each candidate subject: determining, based on the plurality of sets of requisitioned genes and the specific gene, a measure of distance between the candidate subject and the specific gene; and selecting the candidate subject to include in the subset of the plurality of subjects when the measure of distance is at least a threshold distance; and (c) estimating prevalence of the genetic disorder using assay results for the selected subset of the subjects, given the incidence of disease-causing mutations in the specific gene.

In some embodiments, determining the measure of distance between the candidate subject and the specific gene comprises: determining a distance between each set of requisitioned genes of the candidate subject and the specific gene to obtain one or more distances; and determining the measure of distance between the candidate subject and the specific gene based on the one or more distances.

In some embodiments, determining the measure of distance between the candidate subject and the specific gene comprises determining a minimum distance of the one or more distances. In some embodiments, determining a distance between the candidate subject and the specific gene comprises: determining the distance as 0 when the plurality of requisitioned genes of the candidate subject comprises the specific gene; and determining the distance as 1 when (a) the distance is not 0, and (b) the plurality of requisitioned genes of the candidate subject comprises a gene in common with a plurality of requisitioned genes of a second subject.

In some embodiments, determining a distance between the candidate subject and the specific gene comprises: determining the distance as 2 when (a) the distance is not 0 or 1; and (b) the plurality of requisitioned genes of a second subject, the second subject having a distance 1 from the candidate subject, comprises a gene in common with a plurality of requisitioned genes of a third subject.

In some embodiments, the threshold distance is at least 1. In some embodiments, the threshold distance is at least 2. In some embodiments, estimating prevalence of the genetic disorder comprises estimating prevalence of a dominant genetic disorder. In some embodiments, estimating prevalence of a genetic disorder associated with a mutation of a specific gene, comprises estimating prevalence of a genetic disorder associated with a de novo mutation. In some embodiments, estimating prevalence of the genetic disorder comprises estimating prevalence of a recessive genetic disorder. In some embodiments, estimating prevalence of a genetic disorder associated with a mutation of a specific gene, comprises estimating prevalence of a genetic disorder associated with an inherited mutation. In some embodiments, the plurality of gene panel results comprises at least 100,000 gene panel results. In some embodiments, estimating prevalence of the genetic disorder comprises estimating using Hardy-Weinberg equilibrium equation. In some embodiments, estimating prevalence of the genetic disorder comprises estimating in a population of a single sex. In some embodiments, estimating prevalence of the genetic disorder comprises estimating in a single ethnicity.

### BRIEF DESCRIPTION OF THE DRAWINGS

FIGS. 1A-1C show an illustrative system, flowchart, and graph that may be used in estimating prevalence, in a population, of one or more mutations of a specific gene associated with a genetic disorder. FIG. 1A shows an illustrative system 10 for estimating prevalence. FIG. 1B is a flowchart of an illustrative process 100 for estimating prevalence. FIG. 1C is an exemplary graph showing a method of calculating distance between a candidate subject (S1) and other subjects (S2, S3, and S4) based on a specific gene, G2 that may be used when estimating prevalence.

FIG. 2 depicts an illustrative implementation of a computer system that may be used in connection with some embodiments of the technology described herein.

FIG. 3 is a diagram depicting the key features of the Invitae® gene assay results database.

FIGS. 4A-4B show graphs depicting that Invitae's database includes many different disorders and is highly enriched for rare disorders (graph excludes cancers).

FIG. 5 shows the number of individuals sequenced per gene in a database of 3.9M+ patient records.

FIG. 6 shows that prevalence estimates using the Invitae database correlate with those derived from gnomAD. Each point on the graph corresponds to a specific gene.

FIG. 7 shows that Invitae's genetic prevalence for autosomal recessive (AR) genes correlates with literature estimates. Each point on the graph corresponds to a specific gene.

FIG. 8 shows that Invitae's genetic prevalence for autosomal dominant (AD) genes correlates with literature estimates. Each point on the graph corresponds to a specific gene.

### DETAILED DESCRIPTION

A central challenge in estimating the prevalence of one or more disease-causing (aka pathogenic) mutations of a specific gene associated with a genetic disorder is accounting for sampling bias in genomic databases that are used in the estimation process. This is challenging, in part, because subjects who are tested for genetic disorders using genome sequencing typically already have, or are suspected of having, the genetic disorder. This can create a sampling bias. The inventors of this disclosure, recognizing this problem, developed an improved method for estimating prevalence of one or more mutations of a specific gene associated with a genetic disorder. This method may involve reducing or eliminating bias by selecting a subset of data for estimating prevalence of the mutations where the subset does not include subjects specifically tested for the genetic disorder. The method may also include removing subjects from the subset that have a gene panel which shares a gene in common with a gene panel used to test for the genetic disorder.

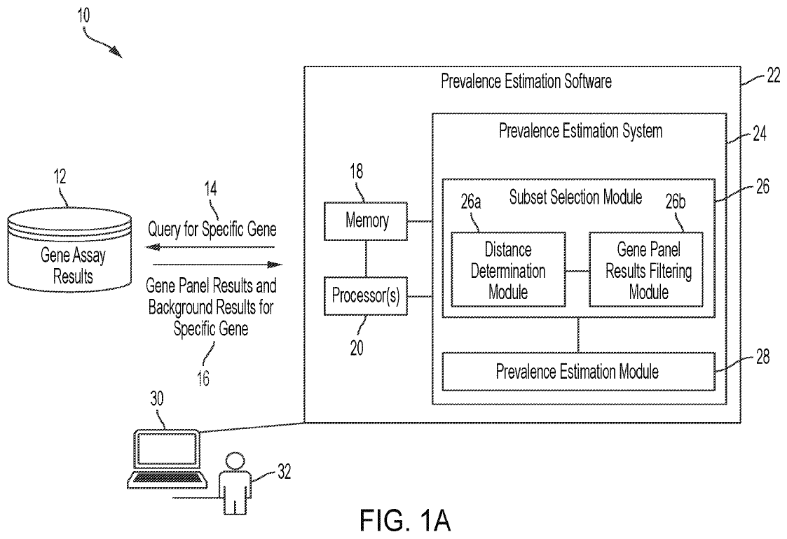

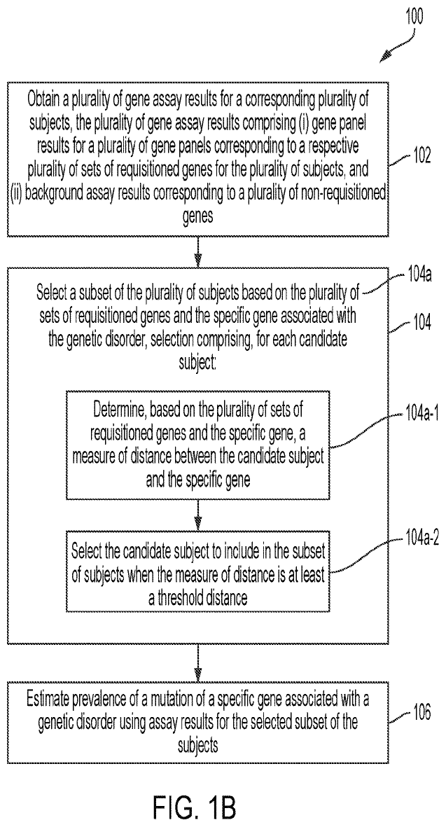

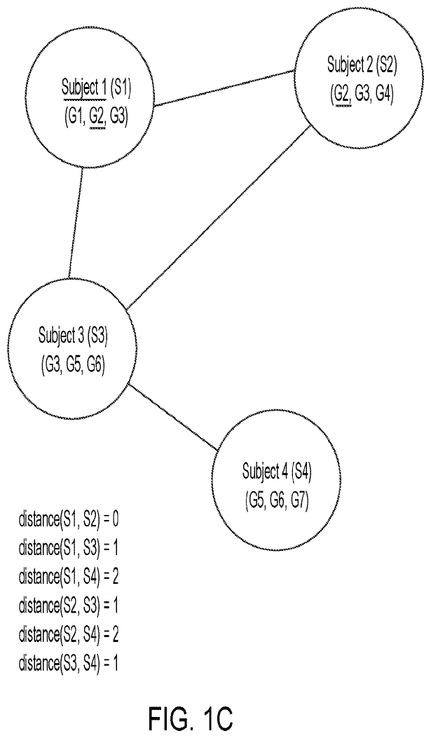

FIG. 1A illustrates an illustrative system 10 that can be used to estimate prevalence of mutations of a specific gene associated with a genetic disorder, in accordance with some embodiments of the technology described herein. As shown in FIG. 1A, system 10 includes a database 12 storing gene assay results, prevalence estimation system 22, and computing device 30. A user 32 can use device 30 to interact with prevalence estimation system 22 in order to estimate prevalence of one or more mutations of a specific gene associated with a genetic disorder. The user may provide input (via a user interface, e.g., a graphical user interface, presented by software executing on computing device 30) to system 22, which input indicates a genetic disorder and a specific gene of interest. In turn, prevalence estimation system 22 may query database 12 (e.g., using query 14) in order to obtain gene panel results and/or background results (from a plurality of subjects) for the specific gene of interest (16). The obtained gene panel and/or background results may be stored in memory 18. The obtained gene panel and background results may be processed using processor(s) 20 when executing instructions associated with prevalence estimation software 24 to estimate prevalence of one or more disease-causing mutations of a specific gene associated with a genetic disorder.

Prevalence estimation software 24 may comprise subset selection module 26. Subset selection module 26 may comprise computer code that, when executed (e.g., using processor(s) 20), identifies a subset of subjects (having corresponding gene panel results and/or background results) with decreased sampling biases. For example, background assay results may comprise results associated with the specific gene (e.g., the sequence of the specific gene) which may be used to in estimating prevalence of the mutation using prevalence estimation software 24. Subset selection module 26 may comprise distance determination module 26a. Distance determination module 26a may comprise computer code that, when executed (e.g. using processor(s) 20), determines a measure of distance between a candidate subject's gene panel results and one or more other subject's gene panel results based on the specific gene. The distance determination may be inputted into the gene panel results filtering module 26b. The gene panel results filtering module 26b may comprise computer code that, when executed (e.g. using processor(s) 20), determines, using the measure of distance, whether the candidate subject should be included in the subset of subjects. The corresponding gene panel results and/or background results of the subset of subjects may then be inputted into prevalence estimation module 28. Prevalence estimation module 28 may comprise computer code, that when executed (e.g. using processor(s) 20), estimates the prevalence of the one or more mutations using the gene panel results and/or the background results associated with the subset of subjects.

The gene assay results database 12 may be obtained from any suitable source. In some embodiments, the gene assay results database 12 comprises gene assay results from a plurality of subjects. In some embodiments, the gene assay results comprise gene panel results and/or background panel results. Gene assay results may be obtained from any suitable source including those described herein in the section entitled, “Obtaining a Plurality of Gene Assay Results.”

In some embodiments, a user 32 may query for specific gene 14 in gene assay results database 12 using computing device 30 in any suitable way. In some embodiments, the query is not from a user. For example, the query may be from another software program, a database, etc. Computing device 30 may be any suitable computing device including those computing devices described in the section entitled “Computer Implementation.” Gene panel results and/or background results for a specific gene 16 may be dependent on the identity of the specific gene. Thus, some gene assay results may be gene panel results for a first specific gene and background results for a second specific gene.

Prevalence estimation system 22 may be any suitable prevalence estimation system. In some embodiments, prevalence estimation system 22 comprises prevalence estimation software 24, processor(s) 20 and memory 18. In some embodiments, prevalence estimation system 22 further comprises a network I/O interface(s), a user I/O interfaces(s), and/or a non-volatile storage. Memory 18 may be any suitable memory. In some embodiments, memory 18 is volatile memory (e.g., random access memory). Memory is described herein including in the section entitled, “Computer Implementation.” Processor(s) 20 may be any suitable processor or may be a plurality of suitable processors including those described herein in the section entitled “Computer Implementation”.

Prevalence estimation software 24 may be any suitable prevalence estimation software. In some embodiments, prevalence estimation software 24 comprises a subset selection module 26 and a prevalence estimation module 28. The prevalence estimation software may comprise two different modules. A first module that may decrease and/or remove biases in the gene panel results and background results for the specific gene 16 (e.g., the subset selection module 26) and a second module that estimates the prevalence of a mutation of the specific gene associated with the genetic disorder (e.g., prevalence estimation module 28) using the output of the first module.

The subset selection module 26 may be any suitable subset selection module. In some embodiments, the subset selection module 26 is computer code that, when executed, selects gene assay results of a subset of subjects for use by the prevalence estimation module 28. In some embodiments, the subset selection module comprises two modules: a distance determination module 26a and a gene panel results filtering module 26b. In some embodiments, the distance determination module 26a comprises computer code that, when executed, calculates a distance between a candidate subject and other subjects of the subjects associated with the obtained gene panel results and background results based on the specific gene. The distance determination module, when executed, may perform any suitable method for determining this distance including those methods described herein in the section entitled, “Selecting a Subset of the Plurality of Subjects.” In some embodiments, the gene panel results filtering module 26b, when executed, determines whether to include the candidate subject in the subset of subjects based on the distance determined using the distance determination module 26a. The gene panel results filtering module 26b, when executed, may perform any suitable method for including candidate subjects in the subset of subjects based on the distance determined using the distance determination module 26a including those methods described herein in the section entitled, “Selecting a Subset of the Plurality of Subjects.”

The prevalence estimation module 26 may be any suitable prevalence estimation module. In some embodiments, the prevalence estimation module, when executed, estimates the prevalence of one or more mutations of a specific gene associated with a genetic disorder using the gene assay results of the subset of subjects from the subset selection module 26. This estimation may be performed using any suitable method including those methods described herein in the section entitled, “Estimating Prevalence of a Mutation Associated with a Genetic Disorder.”

FIG. 1B is a diagram depicting an illustrative process 100 for estimating prevalence, in a population, of one or more mutations of a specific gene associated with a genetic disorder. Process 100 begins at act 102, where a plurality of gene assay results for a corresponding plurality of subjects are obtained. In some embodiments, the plurality of gene assay results comprise: (i) gene panel results for a plurality of gene panels corresponding to a respective plurality of sets of requisitioned genes for the plurality of subjects, and/or (ii) background assay results corresponding to a plurality of non-requisitioned genes. Act 102 may be performed in any suitable way including those described herein in the section entitled “Obtaining a Plurality of Gene Assay Results.”

Process 100 continues at act 104, which comprises acts 104a, 104a-1 and/or 104a-2. Act 104a includes selecting a subset of a plurality of subjects based on the plurality of sets of requisitioned genes and the specific gene associated with the genetic disorder. In some embodiments, act 104a further comprises, for selected subjects of the plurality of subjects (e.g., for each candidate subject), performing acts 104a-1 and 104a-2. Act 104a may be performed in any suitable way including in any of the ways described herein including in the section entitled “Selecting a Subset of the Plurality of Subjects.” In some embodiments, act 104a continues at act 104a-1 to determine, based on the plurality of sets of requisitioned genes and the specific gene, a measure of distance between the candidate subject and the specific gene. Act 104a-1 may be performed in any suitable way including those described herein in the section entitled “Selecting a Subset of the Plurality of Subjects.” Act 104a-1 is often followed by act 104a-2 which includes selecting a candidate subject to include in the subset of subjects when the measure of distance is at least a threshold distance. Act 104a-2 may be performed in any suitable way including those described herein in the section entitled “Selecting a Subset of the Plurality of Subjects.” Acts 104a-1 and 104a-2 can be performed in any order, with FIG. 1B illustrating an exemplary embodiment.

Process 100 may continue at act 106 to estimate prevalence of one or more mutations of a specific gene associated with a genetic disorder using gene assay results for the selected subset of subjects. Act 106 may be performed in any suitable way including those described herein in the section entitled “Estimating Prevalence of a Mutation Associated with a Genetic Disorder.”

Alternatively, in some embodiments, Process 100 does not include act 106. In some embodiments, act 104 is followed by an act that comprises making a different determination using the gene assay results of a subset of subjects. For example, determining whether two different mutations found in the same gene of a given subject are in cis or in trans configuration. This may be done in any suitable way including those described herein in the section entitled “Cis or Trans Determination of Gene Mutations.” In another example, act 104 may be followed by an act that comprises determining whether a mutation is associated with a genetic disorder. This may be done in any suitable way including those described herein in the section entitled, “Associating a Gene Mutation with a Genetic Disorder.” In another example, act 104 is followed by an act that comprises estimating the penetrance of a mutation of a specific gene associated with a genetic disorder. This may be done in any suitable way including those described herein in the section entitled “Estimating Penetrance of a Pathogenic Mutation.”

In some embodiments, this disclosure provides a method for estimating prevalence, in a population, of one or more mutations of a specific gene associated with a genetic disorder.

“Prevalence” may refer to how often one or more mutations (usually, disease-causing mutations) of a specific gene associated with a genetic disorder occurs in a population and/or how often a genetic disorder occurs in a population. Prevalence may be estimated in any suitable way including those described herein in the section entitled “Estimating Prevalence of a Mutation Associated with a Genetic Disorder.” In some embodiments, estimating prevalence comprises estimating prevalence of one or more genetic disorders and/or one or more mutations of one or more specific genes associated with one or more genetic disorders.

A “population” may refer to a set of subjects that share one or more common identifying feature(s). A population may be used to indicate the group of subjects for which one wishes to learn something (e.g., an estimate of the prevalence of one or more mutations of a specific gene associated with a genetic disorder, in an Asian population). In some embodiments, estimating prevalence (e.g., of a mutation or of a genetic disorder) in a population refers to estimating prevalence in the population of all humans. In some embodiments, estimating prevalence in a population refers to estimating prevalence in a population of all living humans. In some embodiments, estimating prevalence in a population refers to estimating prevalence in a population of a given ethnicity. In some embodiments, estimating prevalence in a population refers to estimating prevalence in a population of a given race. In some embodiments, estimating prevalence in a population refers to estimating prevalence in a population of a given sex. In some embodiments, estimating prevalence in a population refers to estimating prevalence in a population of a given geographical location (e.g., a given country).

A “genetic disorder” may refer to a disease or condition caused in whole or in part by a mutation in the genome of a subject. Many genetic disorders are known in the art, e.g., as described in genome.gov/For-Patients-and-Families/Genetic-Disorders, accessed May 16, 2024; Lee C E et al, iScience. 2020 May 22;23(5):101123; Leblond, Claire S., et al., Molecular and Cellular Neuroscience 113 (2021): 103623; and Joanna S. Amberger et al., Nucleic Acids Research, Volume 43, Issue D1, 28 Jan. 2015, Pages D789-D798. For example, Autosomal dominant polycystic kidney disease (ADPKD) is a genetic disorder, which has a later-onset and is due primarily to inherited variants. ADPKD is typically due to a heterozygous, disease-causing mutation in PKD1, PKD2, or to a lesser extend ALG5, ALG9, DNAJB11, GANAB, IFT140. Typically, APDKD is acquired via inheritance of an autosomal dominant disease-causing mutation in PKD1 or PKD2. However, 10%-20% of individuals with ADPKD have the disorder due to a de novo disease-causing mutation. Harris P C et al., Polycystic Kidney Disease, Autosomal Dominant. 2002 Jan. 10 [Updated 2022 Sep. 29]. GeneReviews® [Internet]. Seattle (WA): University of Washington, Seattle; 1993-2024. An epidemiological study also suggests that ADPKD prevalence differs by ethnicity: Prevalence (per 100,000) of ADPKD was lower in Hispanic (39.9) and Asian (48.9) patients compared to Black (73.0) and (non-Hispanic) White (63.2) patients. Aung T T, et al., Kidney 360. 2021 Sep. 22;2(12):2010-2015. Given that the prevalence varies by ethnicity and that it is mostly inherited, this suggests that the prevalence per variant in ADPKD also varies.

In some embodiments, estimating prevalence, in a population, of a mutation of a specific gene associated with a genetic disorder, comprises estimating prevalence of a mutation associated with recessive genetic disorder. In some embodiments, estimating prevalence, in a population, a mutation of a specific gene associated with a genetic disorder, comprises estimating prevalence of a mutation associated with dominant genetic disorder. In some embodiments, estimating prevalence, in a population, of a mutation of a specific gene associated with a genetic disorder, comprises estimating prevalence of a mutation associated with an autosomal recessive genetic disorder. In some embodiments, estimating prevalence, in a population, of a mutation of a specific gene associated with a genetic disorder, comprises estimating prevalence of a mutation associated with a sex-linked recessive genetic disorder. In some embodiments, estimating prevalence, in a population, of a mutation of a specific gene associated with a genetic disorder, comprises estimating prevalence of a mutation associated with an autosomal dominant genetic disorder. In some embodiments, estimating prevalence, in a population, of a mutation of a specific gene associated with a genetic disorder, comprises estimating prevalence of mutation associated with a sex-linked dominant genetic disorder.

In some embodiments, estimating prevalence, in a population, of a mutation of a specific gene associated with a genetic disorder, comprises estimating prevalence of a mutation associated with a cancer. In some embodiments, estimating prevalence, in a population, of a mutation of a specific gene associated with a genetic disorder, comprises estimating prevalence of a mutation associated with cardiological genetic disorder, a neurological genetic disorder, a pediatric genetic disorder, a metabolic genetic disorder, an immunological genetic disorder, an ophthalmological genetic disorder, hematological genetic disorder, or a dermatological gene disorder. In some embodiments, estimating prevalence, in a population, of a mutation of a specific gene associated with a genetic disorder, comprises estimating prevalence of a mutation associated with a neurological disorder.

In some embodiments, the method comprises estimating a prevalence of plurality of mutations of a specific gene associated with a genetic disorder (e.g., estimating using the subset of subjects). In some embodiments, the method comprises estimating a prevalence of plurality of mutations of a plurality of specific genes associated with a genetic disorder (e.g., estimating using the subset of subjects). In some embodiments, the method comprises identifying a mutation with the highest prevalence of the one or more mutations associated with the genetic disorder.

A genetic disorder may be associated with a mutation in a specific gene when the mutation in the specific gene is indicative of the genetic disorder. A mutation in the specific gene may be present in a subject's genome when the subject has the genetic disorder. A genetic disorder-associated mutation that is present in a specific gene may be a cause of the genetic disorder. A genetic disorder-associated mutation may be a dominant mutation, meaning that only one of the two copies of a subject's gene needs to be mutated in order for the individual to develop the genetic condition. A genetic disorder-associated mutation may be a recessive mutation, meaning that both copies of a subject's gene are required to be mutated in order for the individual to develop the genetic condition. Associations between genetic disorders and mutations in specific genes are often known, e.g., as described in Lee C E et al., iScience. 2020 May 22;23(5):101123; Leblond, Claire S., et al., Molecular and Cellular Neuroscience 113 (2021): 103623; Joanna S. Amberger et al., Nucleic Acids Research, Volume 43, Issue D1, 28 Jan. 2015, Pages D789-D798; and Stenson, P. D., et al., Hum Genet 139, 1197-1207 (2020).

A “mutation” may refer to a change in the genome of an organism (e.g., the genome of a subject). In some embodiments, a mutation refers to a change in the genome of an organism (e.g., a human) relative to a reference genome (e.g., T2T-CHM13, GRCh38, or GRCh37). In some embodiments, the mutation is an inherited mutation. In some embodiments, the mutation is an acquired (i.e., de novo) mutation. In some embodiments, the mutation is a single nucleotide variant or polymorphism, an insertion, a deletion, a frameshift mutation, transversion, a transposition, a variation in the copy number of a gene or gene sequence (CNV), or a combination thereof. In some embodiments, the mutation is a pathogenic mutation (e.g., the mutations is a cause of a genetic disorder).

A “specific gene” may refer to a gene that is associated with a genetic disorder (e.g., a pathogenic mutation in the specific gene is a cause of the genetic disorder). In some embodiments, a “specific gene” may refer to a gene that has been selected for use in estimating the prevalence of a genetic disorder in a population.

In some embodiments, this disclosure provides a method for estimating prevalence, in a population, of a genetic disorder using the prevalence of a mutation of a specific gene associated with the genetic disorder. In some embodiments, this disclosure provides a method for estimating prevalence, in a population, of a genetic disorder using prevalence estimates of a plurality of mutations of a specific gene associated with the genetic disorder. In some embodiments, this disclosure provides a method for estimating prevalence, in a population, of a genetic disorder using prevalence estimates of a plurality of mutations of a plurality of specific genes associated with the genetic disorder. For example, estimating prevalence of a genetic disorder may comprise estimating using mutations of two or more specific genes that are associated with the genetic disorder.

In some embodiments, the disclosure provides a method comprising: (i) determining whether a mutation of a given gene is associated with a genetic disorder (e.g., using any suitable method including those methods described herein in the section entitled “Associating a Gene Mutation with a Genetic Disorder”); if the mutation is associated with the genetic disorder (ii) estimating prevalence, in a population, of the mutation (e.g., using any suitable method including the methods described in the sections entitled “Obtaining a Plurality of Gene Assay Results”, “Selecting a Subset of the Plurality of Subjects” and “Estimating Prevalence of a Mutation Associated with a Genetic Disorder”). In some embodiments, the method further comprises (iii) estimating the prevalence of the genetic disorder using the estimated prevalence of the mutation. In some embodiments, the method comprises performing (i)-(ii) (optionally (i)-(iii)) for a plurality of mutations of a given gene. In some embodiments, the method comprises performing (i)-(ii) (optionally (i)-(iii)) for a plurality of mutations of a plurality of given genes.

### Obtaining a Plurality of Gene Assay Results

In some embodiments, the method comprises obtaining a plurality of gene assay results for a corresponding plurality of subjects, the plurality of gene assay results comprising: (i) gene panel results for a plurality of gene panels corresponding to a respective plurality of sets of requisitioned genes for the plurality of subjects, and (ii) background assay results corresponding to a plurality of non-requisitioned genes.

A “gene assay result” may refer to a result from an assay that was used to sequence a plurality of genes (e.g., genes associated with a genetic disorder) of a subject. In some embodiments, a gene assay result comprises a gene panel result. A “gene panel result” may refer to a result from sequencing a set of requisitioned genes. A “set of requisitioned genes” comprises one or more genes that are selected for use in identifying a genetic disorder in a subject (e.g., genes that are known to be associated with the genetic disorder being tested for using the gene panel). A set of requisitioned genes is also referred to as a gene panel. In some embodiments, a plurality of sets of requisitioned genes refers to at least 2 sets of requisitioned genes (e.g., at least 3 sets of requisitioned genes, at least 4 sets of requisitioned genes, at least 5 sets of requisitioned genes, at least 10 sets of requisitioned genes, or at least 20 sets of requisitioned genes).

In some embodiments, a gene assay result comprises a plurality of gene panel results. For example, a gene assay result may comprise a plurality of gene panel results when a subject is tested for a plurality of different genetic disorders. In some embodiments, a gene assay result comprises results from at least 1 gene panel (e.g., at least 2 gene panels, at least 3 gene panels, at least 4 gene panels, at least 5 gene panels, at least 10 gene panels, or at least 20 gene panels). In some embodiments, the gene assay result comprises a background assay result. A “background assay result” may refer to a result from sequencing one or more non-requisitioned genes of a subject (e.g., a subject that is being tested for a genetic disorder using a gene panel). “Non-requisitioned genes” may refer to genes that were not selected for use in identifying a genetic disorder in a given subject (e.g., genes that are not known to be associated with the genetic disorder being tested for using the gene panel). Background assay results comprising results of a specific gene may be used in estimating the prevalence of one or more mutations of the specific gene associated with a genetic disorder. In some embodiments, the gene assay result comprises a gene panel result and a background assay result.

In some embodiments, a gene assay result may comprise sequences of the genes sequenced using the gene assay. In some embodiments, a gene assay result may comprise an indication of the sequences of the genes sequenced using the gene assay. The indication may indicate whether a given gene comprises a mutation. When a gene comprises mutation, the indication may indicate the location of the mutation in the gene and/or the type of mutation. When a gene comprises a mutation, the indication may indicate whether the mutation is known to be associated with the genetic disorder being assayed for using the gene panel. In some embodiments, the indication is a numeric code. In some embodiments, the indication is an alphabetic code. In some embodiments, the indication is an alphanumeric code. In some embodiments, the indication is an alphabetic string. In some embodiments, the gene assay result comprises results for at least 5 genes (e.g., at least 10 genes, at least 50 genes, at least 100 genes, at least 500 genes, at least 1000 genes, at least 2500 genes, at least 5000 genes, at least 10,000 genes, at least 15,000 genes, or at least 20,000 genes).

“Gene panel results” may refer to a result from a plurality of gene assays (i.e., a plurality of gene assay results). In some embodiments, gene panel results comprise results from at least 100 genes assays (e.g., at least 500 genes assays, at least 1,000 genes assays, at least 10,000 genes assays, at least 100,000 genes assays, at least 1,000,000 genes assays, or at least 10,000,000 genes assays). In some embodiments, fewer than 100 gene assays may be used without deviating from the scope of the methods described and/or claimed herein.

In some embodiments, the gene assay results correspond to a plurality of subjects. A “subject” refers to a multi-cellular organism. In some embodiments, the subject is mammal. In some embodiments, the subject is a human. In some embodiments, the subject is a non-human primate. In some embodiments, the subject is a mouse. In some embodiments, a plurality of subjects comprises at least 2 (e.g., at least 5 subjects, at least 10 subjects, at least 50 subjects, at least 100 subjects, at least 1000 subjects, at least 10,000 subjects, at least 100,000 subjects, or at least 1,000,000 subjects). In some embodiments, each gene assay result of the gene assay results corresponds to a different subject. In some embodiments, one or more gene assay results of the gene assay results correspond to the same subject.

Obtaining gene assay results comprises obtaining gene assay results in any suitable matter. In some embodiments, obtaining gene assay results comprises generating gene assay results (e.g., by sequencing genomic material of a subject). In some embodiments, obtaining gene assay results comprises obtaining gene assay results from one or more databases of gene assay results. In some embodiments, obtaining gene assay results comprises obtaining gene assay results from an Invitae database of gene assay results. In some embodiments, obtaining gene assay results comprises obtaining gene assays results from local hardware storage. In some embodiments, obtaining gene assay results comprises obtaining gene assay results from cloud storage. In some embodiments, obtaining gene assay results comprises obtaining gene assay results from distributed storage.

### Selecting a Subset of the Plurality of Subjects

In some embodiments, the method comprises selecting a subset of a plurality of subjects based on a plurality of sets of requisitioned genes for each of the plurality of subjects and a specific gene associated with a genetic disorder of interest, the selecting comprising, for each candidate subject of the plurality of subjects: determining a distance between each gene in a set of requisitioned genes of the candidate subject and the specific gene of interest to obtain one or more distances; and selecting the candidate subject to include in the subset of subjects when the measure of distance is at least a threshold distance. The subset of the plurality of subjects is also referred to as the subset of subjects.

A “candidate subject” may refer to a subject of a plurality of subjects on which a measure of distance is being made to determine if the candidate subject is to be included in the subset of subjects.

A “measure of distance” may refer to a metric that is indicative of the potential sampling bias associated with a gene assay result of a subject (e.g., the candidate subject). In some embodiments, the measure of distance is a positive integer. In some embodiments, the smaller the positive integer, the more bias is associated with the gene assay result of the candidate subject.

A measure of distance may be determined in any suitable way. In some embodiments, determining a measure of distance comprises determining one or more distances between a candidate subject and a specific gene (e.g., when one or more gene panels are associated with the subject). In some embodiments, determining a measure of distance comprises determining one or more distances between a candidate subject and a specific gene and then selecting the minimum of the distances, mean of the distances, median of the distances, mode of the distances, or range of the distances as the measure of distance. In some embodiments, determining a measure of distance comprises determining one or more distances between a candidate subject and a specific gene and then selecting the minimum distance of the distances as the measure of distance.

In some embodiments, determining a measure of distance comprises determining one or more distances based on the proximity of a candidate subject to a specific gene in a graph. In such a graph, the nodes represent subjects and their corresponding requisitioned genes with any one node representing a candidate subject, and the edges between the nodes indicate that nodes have at least one of their requisitioned genes in common. In some embodiments, determining a distance comprises determining the number of edges between the node of a candidate subject (candidate node) and a different node. For example, a first subject (S1) has a corresponding gene panel having requisitioned genes (G1, G2 and G3), a second subject (S2) has a corresponding gene panel having requisitioned genes (G2, G3, G5); a third subject (S3) has a corresponding gene panel having requisitioned genes (G3, G5 and G6); and a fourth subject (S4) has a corresponding gene panel having requisitioned genes (G5, G6 and G7) (see FIG. 1C). Also notated:
S1: (G1, G2, G3);
S2: (G2, G3, G5);
S3: (G3, G5, G6);
S4: (G5, G6 and G7).

In this example, S1 is the candidate subject and G2 is the specific gene. S1 and S2 are separated by one edge and both have the specific gene, G2. Thus, S1 and S2 have a minimum distance in the graph (represented as a distance of 0).

S1 and S3 are also separated by one edge but this edge is because of G3, which is not the specific gene. Thus, S1 and S3 have a distance of 1.

S1 and S4 are separated by two edges; S1 to S3 (G3 in common) and S3 to S4 (G5 and G6 in common), or S1 to S2 (G2 in common) and S2 to S4 (G5 in common). Thus, S1 and S4 have a distance of 2.

To generalize, in this example, the distance equals 0 when the candidate node and the other node are separated by one edge and share the specific gene. The distance equals the number of edges between the candidate node and the other node when the nodes do not share the specific gene.

In some embodiments, determining a distance between the candidate subject and the specific gene comprises determining the distance as 0 when a plurality of requisitioned genes of the candidate subject comprises the specific gene.

In some embodiments, determining a distance between the candidate subject and the specific gene comprises determining the distance as 1 when (a) the distance is not 0, and (b) the plurality of requisitioned genes of the candidate subject comprises a gene in common with a plurality of requisitioned genes of a second subject.

In some embodiments, determining a distance between the candidate subject and the specific gene comprises determining the distance as 2 when (a) the distance is not 0 or 1; and (b) the plurality of requisitioned genes of a second subject, the second subject having a distance 1 from the candidate subject, comprises a gene in common with a plurality of requisitioned genes of the third subject.

Other suitable methods (e.g., hash table(s) and the like) can be used to determine or analyze the measure of distance between the plurality of subjects and the specific gene as disclosed herein without deviating from the scope of the methods described and/or claimed herein.

In some embodiments, the method comprises selecting the candidate subject to include in the subset of subjects when the measure of distance is at least a threshold distance. In some embodiments, a threshold distance is at least 1 (e.g., at least 2, at least 3, at least 4, or at least 5). In some embodiments, a threshold distance is at least 1. In some embodiments, a threshold distance is at least 2. In some embodiments, a threshold distance is 1, 2, 3, 4, or 5. In some embodiments, a threshold distance is 1. In some embodiments, a threshold distance is 2.

In some embodiments, the method comprises selecting a subset of a plurality of subjects based on a plurality of sets of requisitioned genes for each of the plurality of subjects and a plurality of specific genes associated with a genetic disorder of interest, the selecting comprising, for each candidate subject of the plurality of subjects: (a) determining a distance between each gene in a set of requisitioned genes of the candidate subject and each gene of the plurality of specific gene to obtain one or more distances; (b) determining a measure of distance using the one or more distances and (c) selecting the candidate subject to include in the subset of subjects when the measure of distance is at least a threshold distance. In some embodiments, determining a measure of distance comprises selecting the minimum of the distances, mean of the distances, median of the distances, mode of the distances, or range of the distances as the measure of distance.

### Estimating Prevalence of a Mutation Associated With a Genetic Disorder

In some embodiments, the method comprises estimating prevalence, in a population, of a mutation of a specific gene associated with a genetic disorder using gene assay results for the selected subset of the subjects. In some embodiments, estimating prevalence of a mutation of a specific gene associated with a genetic disorder comprises estimating prevalence in any suitable matter. In some embodiments, estimating prevalence of a specific gene associated with a genetic disorder comprises estimating using the Hardy-Weinberg Equilibrium Equation, which is known, e.g., as described in Foundations of Mathematical Genetics Cambridge University Press, Jan. 13, 2000. In some embodiments, estimating prevalence of a specific gene associated with a genetic disorder comprises estimating using the Hardy Weinburg Equilibrium Equation and accounting for departures from this equilibrium e.g., using the Chi-Square Goodness-of-Fit Test, the Hardy-Weinberg Exact Test, and/or a Hardy-Weinberg Proportion Test as described in Wang J et al., Statistical human genetics: methods and protocols (2012): 77-102). In some embodiments, estimating prevalence of a mutation of a specific gene associated with a genetic disorder comprises estimating based on the number of times the mutation occurs in a sample of the population (e.g., gene assay results from a subset of subjects) and the total size of the sample. Other methods of estimating prevalence may be used without deviating from the scope of the methods described and/or claimed herein.

In some embodiments, estimating prevalence of a specific gene associated with a genetic disorder comprises estimating using gene assay results of non-requisitioned genes of the selected subset of subjects. In some embodiments, the method comprises estimating the prevalence of a genetic disorder based on the prevalence of one or more mutations of a specific gene associated with the genetic disorder.

In some embodiments, estimating the prevalence of a mutation of a specific gene associated with a genetic disorder comprises estimating the prevalence of a mutation that is associated with a dominant genetic disorder. A “dominant genetic disorder” may refer to a genetic disorder that requires only one abnormal (e.g., mutated) allele to cause the genetic disorder. An “autosomal dominant genetic disorder” may refer to a genetic disorder that requires only one mutated allele in an autosome to cause the genetic disorder. Dominant genetic disorders are known, e.g., as described online in the Encyclopedia Britannica (www.britannica.com/science/human-genetic-disease/Prenatal-diagnosis, accessed May 17, 2024). In some embodiments, the dominant genetic disorder is Huntington's disease, Marfan syndrome, or achondroplasia. In some embodiments, estimating prevalence of the genetic disorder comprises estimating the prevalence of a dominant genetic disorder.

In some embodiments, estimating the prevalence of a mutation of a specific gene associated with a genetic disorder comprises estimating the prevalence of a de novo mutation that is associated with the genetic disorder. A “de novo” mutation refers to a mutation in a subject (e.g., a human) that was not inherited from the subject's parents. De novo mutations that cause genetic disorders typically cause dominant genetic disorders. De novo mutations that are associated with genetic disorders are known, e.g., as described in Acuna-Hidalgo, R. et al., Genome Biol 17, 241 (2016). In some embodiments, estimating prevalence of the genetic disorder associated with a de novo mutation comprises estimating the prevalence of the de novo mutation.

In some embodiments, estimating prevalence of a mutation of a specific gene associated with a genetic disorder comprises estimating prevalence of a mutation associated with a recessive genetic disorder. A “recessive genetic disorder” may refer to a disorder caused by two abnormal (e.g., mutated) alleles of a gene. An “autosomal recessive genetic disorder” may refer to a disorder caused by two abnormal alleles of a gene, where the alleles are in an autosome. Recessive genetic disorders are typically inherited when each parent has at least 1 recessive allele. Recessive genetic disorders are known, e.g., as described in Stenson, P. D., et al., Hum Genet 139, 1197-1207 (2020). In some embodiments, a recessive genetic disorder is cystic fibrosis, sickle cell anemia, or Tay-Sachs disease. In some embodiments, estimating prevalence of a recessive genetic disorder comprises estimating the prevalence of a mutation of a specific gene associated with the recessive genetic disorder.

In some embodiments, estimating prevalence of a mutation of a specific gene associated with a genetic disorder comprises estimating prevalence of an inherited mutation that is associated with the genetic disorder. An “inherited mutation” may refer to a mutation in a subject that was also present in the germline of a parent (or both parents) of the subject. Inherited mutations are typically associated with recessive genetic disorders, or later-onset dominant genetic disorders. For example, inherited mutations that cause cystic fibrosis may be mutations in the cystic fibrosis transmembrane conductance regulator (CFTR) gene (e.g., a F508del mutation). In another example, inherited mutations that cause sickle cell disease may be mutations in the beta-globin gene (e.g., E6V mutation). In some embodiments, estimating prevalence of a genetic disorder comprises estimating the prevalence of an inherited mutation of a specific gene associated with the genetic disorder.

### Cis or Trans Determination of Gene Mutations

A pair of mutations in a gene can occur in cis (i.e., same allele) or trans (i.e., different alleles), and the mutations can sometimes be damaging to gene function (e.g., loss-of-function) or can be neutral. These distinctions can have important consequences when determining the potential of a pair of mutations to cause an autosomal recessive or X-linked recessive disease when, for example, both copies of a gene carry a mutation (e.g., in trans) and the mutations are damaging. In cis and in trans mutation pairs are often indistinguishable when using short read sequencing data, for example when two mutations are too far apart to be captured on the same short read.

Conventional methods used by genetic diagnostics labs often use short-read sequencing. When a patient sample is found to have two different pathogenic mutations in a gene using short-read technology, it is often difficult to unambiguously establish a positive molecular diagnosis. In the above example, a positive molecular diagnosis is only possible when the two mutations (e.g., pathogenic mutations) are determined to be in trans. Typically, such a diagnosis can often be confirmed by testing one or both of the parents. However, this is expensive and sometimes not feasible. The absence of a definitive positive molecular diagnosis often deprives a subject of a genetic explanation for their condition and also can preclude a subject from taking part in a clinical trial with inclusion criteria requiring a positive molecular diagnosis.

The inventors recognized that the gene assay data from the subset of subjects described above can be used to help determine whether two gene mutations are in cis or in trans in a given subject (also referred to as phasing). If two gene mutations in a dataset are observed together in an unbiased population (e.g., the selected subset of subjects) constructed for a specific gene, then it is likely that this combination of mutations is observed in unaffected individuals, and therefore, that these two mutations likely appear in cis in the population. As part of a computational model that determines a cis or in trans configuration, this piece of evidence would weigh in favor of a cis configuration for a new patient. In contrast, if the two mutations are only observed in different individuals in the unbiased population, this would weigh in favor of a trans configuration in a new patient.

In some embodiments, this disclosure provides a method of determining whether two mutations of a specific gene are likely to occur in cis or in trans in a subject having or suspected of having a genetic disorder associated with mutation of the specific gene. Two mutations may occur in cis when the two mutations are found on the same allele of a subject. Two mutations may occur in trans when they are found on different alleles of the subject.

In some embodiments, a method of determining whether two mutations of a specific gene are likely to occur in cis or in trans in a subject having or suspected of having a genetic disorder associated with mutation of the specific gene comprises: (a) obtaining a plurality of gene assay results for a corresponding plurality of subjects, each gene assay result comprising (i) gene panel results corresponding to a plurality of requisitioned genes and (ii) background results corresponding to a plurality of non-requisitioned genes; (b) selecting a subset of the subjects based on the plurality of requisitioned genes and the specific gene, the selecting comprising, for each candidate subject: determining a measure of distance between the candidate subject and the specific gene; and selecting the candidate subject to include in the subset of subjects when the measure of distance is at least a threshold distance. Acts (a) and (b) may be performed in any suitable way including those described herein in the sections entitled “Obtaining a Plurality of Gene Assay Results” and “Selecting a Subset of the Plurality of Subjects.”

In some embodiments, the method comprises identifying the two mutations of the specific gene in the gene assay results of the selected subset of the subject. Identifying the two mutations of the specific gene in the gene assay results of the selected subset of subjects may be performed in any suitable manner.

In some embodiments, the method comprises determining, using the two mutations identified, whether the two mutations of a specific gene are likely to occur in cis or in trans in the subject. In some embodiments, the determining comprises determining whether the two mutations are found in the same subject of the subset of subjects. In some embodiments, the determining comprises determining how many times the two mutations are found in the same subject of the subset of subjects. In some embodiments, the determining comprises determining whether the two mutations are found in different subjects of the subset of subjects.

In some embodiments, the method comprises determining, using the two mutations identified, that the two mutations are likely to occur in cis in the subject when the two mutations are found in the same gene assay result of the gene assay results at least a threshold number of times. In some embodiments, the method comprises determining, using the two mutations of the specific gene, that the two mutations of the specific gene are likely to occur in trans in the subject when the two mutations are not found in the same gene assay result of the plurality of gene assay results at least a threshold number of times. In some embodiments, the threshold is at least 1 gene assay result. In some embodiments, the threshold is at least 2 gene assay results. In some embodiments, the threshold is at least 3 gene assay results. In some embodiments, the threshold is at least 4 gene assay results. In some embodiments, the threshold is at least 5 gene assay results. In some embodiments, the threshold is 1, 2, 3, 4, or 5 gene assay results. In some embodiments, the threshold is at least 0.01% of gene assay results that comprise the two mutations. In some embodiments, the threshold is at least 0.1% of gene assay results that comprise the two mutations. In some embodiments, the threshold is at least 1% of gene assay results that comprise the two mutations. In some embodiments, the threshold is at least 5% of gene assay results that comprise the two mutations. In some embodiments, the threshold is at least 10% of gene assay results that comprise the two mutations. In some embodiments, the threshold is 0.01%, 0.1%, 1%, 5%, or 10% of gene assay results that comprise the two mutations. In some embodiments, determining whether the two mutations are likely to occur in cis or in trans further comprises determining, using other features (e.g., physical distance between the two variants and linkage disequilibrium, or kinship (family relationship) between the individuals with these mutations), a probability that the two mutations are in trans or in cis in a given individual.

### Associating a Gene Mutation With a Genetic Disorder

A central challenge in analyzing genome sequencing data is determining whether a given mutation observed in the sequencing data is associated with a genetic disorder. For example, determining whether a given mutation causes a genetic disorder in the subject (e.g., the mutation is pathogenic). In some embodiments, this disclosure provides a method of determining whether a mutation in a given gene is associated with a genetic disorder. In some embodiments, the method may operate as follows: if a mutation in a given gene is observed in an “unbiased sample” (e.g., the selected subset of subjects) the same amount or more often (percent-wise for example) than in a sample that seeks testing for the genetic disorder (e.g., subjects whose gene panel was used to test for the disorder), then this favors the mutation being benign (not associated with the genetic disorder). In another example, if a mutation in a given gene is observed more often in the sample that seeks testing for the genetic disorder than the unbiased sample, then this favors a pathogenic mutation (e.g., the mutation is associated with the genetic disorder).

In some embodiments, this disclosure provides a method of determining whether a mutation of a given gene is associated with a genetic disorder, the method comprising: (a) obtaining a plurality of gene assay results for a corresponding plurality of subjects, each gene assay result comprising (i) gene panel results corresponding to a plurality of requisitioned genes and (ii) background results corresponding to a plurality of non-requisitioned genes; (b) selecting a first subset of the subjects based on the plurality of requisitioned genes and the given gene, the selecting comprising, for each candidate subject: determining a measure of distance between the candidate subject and the given gene; and selecting the candidate subject to include in the first subset of subjects when the measure of distance is at least a threshold distance; (c) selecting a second subset of the subjects, the selecting comprising, for each candidate subject: selecting the candidate subject to include in the second subset of subjects when the candidate subject has, is suspected of having, or is being tested for the genetic disorder; and (d) determining whether the mutation is associated with the genetic disorder using gene assay results of the first subset of subjects and gene assay results of the second subset of subjects.

Acts (a) and (b) may be performed in any suitable way including those described herein in the section entitled “Obtaining a Plurality of Gene Assay Results” and “Selecting a Subset of the Plurality of Subjects.” In some embodiments, selecting the first subset of subjects comprises selecting using the same methods as used when selecting the subset of subjects (where the specific gene is replaced by the given gene).

In some embodiments, selecting the second subset of subjects comprises selecting a candidate subject to include in the second subset of subjects when the candidate subject has the genetic disorder (e.g., as determined genetically or by phenotype). In some embodiments, selecting the second subset of subjects comprises selecting the candidate subject to include in the second subset of subjects when the candidate subject is suspected of having the genetic disorder (e.g., based on genetic or phenotypic information). In some embodiments, selecting the second subset of subjects comprises selecting the candidate subject to include in the second subset of subjects when the candidate subject is being tested for the genetic disorder. The candidate subject may be tested for the genetic disorder in any suitable manner including being tested by sequencing genes of a gene panel. In some embodiments, the gene panel of the candidate subject does not comprise the given gene and the background genes of the gene assay corresponding to the gene panel that does comprise the given gene.

In some embodiments, determining whether a mutation is associated with the genetic disorder using the gene assay results of the first subset of subjects and the gene assay results of the second subset of subjects comprises determining how often (e.g., as a frequency, fraction, or percentage) the mutation is observed in the first set of subjects and the second set of subjects. In some embodiments, the method comprises determining a first frequency of the mutation in the first subset of subjects and a second frequency of the mutation in the second subset of subjects. In some embodiments, the method comprises determining that the mutation is associated with the genetic disorder when the second frequency is greater than the first frequency. In some embodiments, the method comprises determining that the mutation is not associated with the genetic disorder when the second frequency is less than or equal to the first frequency. In some embodiments, the method further comprises determining whether a mutation is associated with the genetic disorder using other factors in addition to mutations frequency (e.g., case reports, in vitro studies, and structure-based algorithms).

### Estimating Penetrance of a Pathogenic Mutation

In some embodiments, this disclosure provides a method of estimating the penetrance of a pathogenic mutation in a specific gene associated with a genetic disorder. “Penetrance” may refer to the probability of a subject having or developing a particular phenotype (e.g., a genetic disorder) given a mutation in a gene of a subject (e.g., a mutation in a specific gene associated with a genetic disorder). A “pathogenic mutation” may refer a mutation that is a cause of a genetic disorder in at least some subjects.

In some embodiments, the method comprises: using a computer hardware processor to perform: (a) obtaining a plurality of gene assay results for a corresponding plurality of subjects, the plurality of gene assay results comprising (i) gene panel results for a plurality of gene panels corresponding to a respective plurality of sets of requisitioned genes for the plurality of subjects, and (ii) background assay results corresponding to a plurality of non-requisitioned genes. (a) may be performed in any suitable way including those described in the section entitled “Obtaining a Plurality of Gene Assay Results.”

In some embodiments, the method further comprises: (b) selecting a subset of the plurality of subjects based on the plurality of sets of requisitioned genes and the specific gene associated with the genetic disorder, the selecting comprising, for each candidate subject: determining, based on the plurality of sets of requisitioned genes and the specific gene, a measure of distance between the candidate subject and the specific gene; and selecting the candidate subject to include in the subset of the plurality of subjects when the measure of distance is at least a threshold distance. (b) may be performed in any suitable way including those described in the section entitled “Selecting a Subset of the Plurality of Subjects”.

In some embodiments, the method further comprises (c) estimating the penetrance of the mutation using the gene assay results of the subset of the plurality of subjects. Prevalence may be estimated in any suitable way. In some embodiments, estimating prevalence comprises estimating based on the frequency of the mutation in the subset of subjects. For example, if 10% of subjects of the subset of subjects have the pathogenic mutation, then penetrance is 90% (since the 10% are assumed to not carry the disease). In some embodiments, the method further comprises estimating prevalence using other factors in addition to mutations frequency (e.g., subject age, genetic background factors, and/or environmental factors).

### Computer Implementation

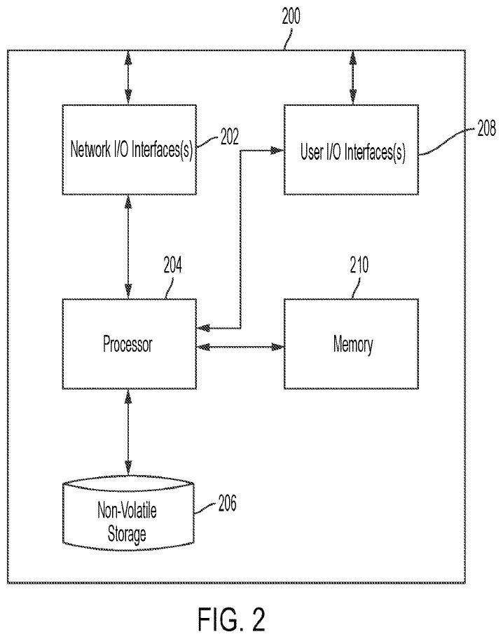

An illustrative implementation of a computer system 200 that may be used in connection with any of the embodiments of the technology described herein (e.g., such as the process of FIG. 1) is shown in FIG. 2. The computer system 200 includes one or more processors 204 and one or more articles of manufacture that comprise non-transitory computer-readable storage media (e.g., memory 210 and one or more non-volatile storage media 206). The processor 204 may control writing data to and reading data from the memory 210 and the non-volatile storage device 206 in any suitable manner, as the aspects of the technology described herein are not limited to any particular techniques for writing or reading data. To perform any of the functionality described herein, the processor 204 may execute one or more processor-executable instructions stored in one or more non-transitory computer-readable storage media (e.g., the memory 210), which may serve as non-transitory computer-readable storage media storing processor-executable instructions for execution by the processor 204.

Computer system device 200 may also include a network input/output (I/O) interface 202 via which the computer system may communicate with other computing devices (e.g., over a network), and may also include one or more user I/O interfaces 208, via which the computer system may provide output to and receive input from a user. The user I/O interfaces 208 may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

In this respect, it should be appreciated that one implementation of the embodiments described herein comprises at least one non-transitory computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments (e.g., part of or all of the processes described above with reference to FIGS. 1A, 1B and 1C). The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. In some embodiments, computer readable media may be non-transitory media.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey a relationship between the fields. However, any suitable mechanism may be used to establish a relationship between information in fields of a data structure, including through the use of pointers, tags or other mechanisms that establish a relationship between data elements.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

### EXAMPLES

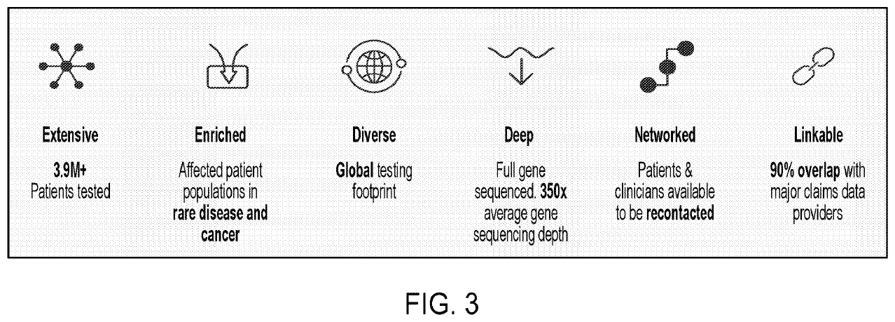

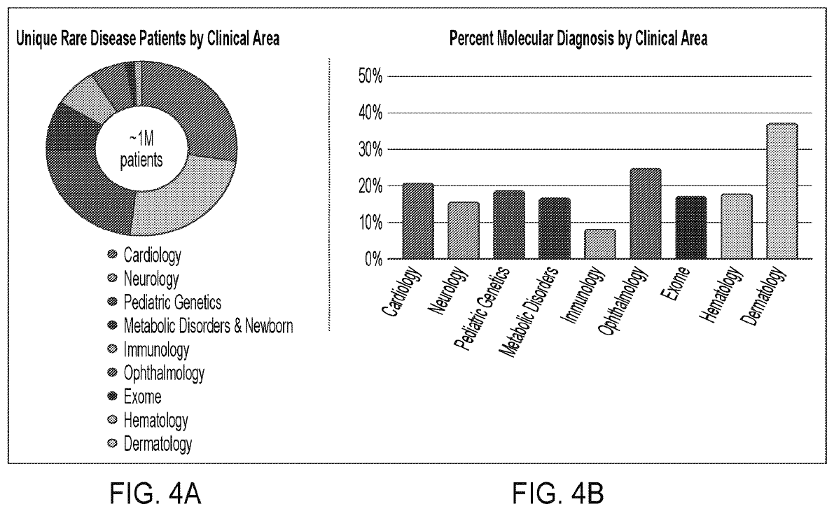

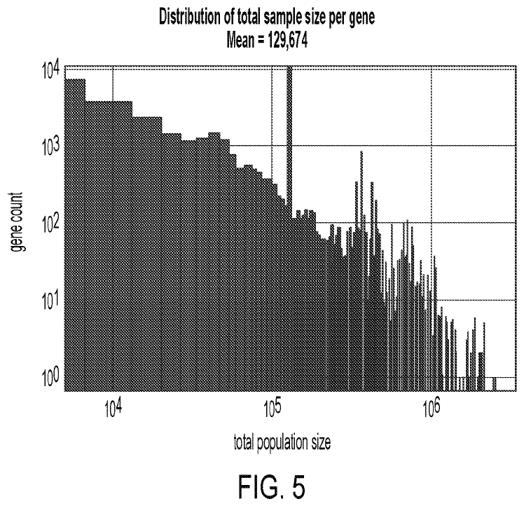

Laboratories may provide clinical testing for many patients, and may maintain and/or have access to data (in, for example, databases) including a large number (e.g., hundreds of thousands, millions, billions) of patient records. For example, Invitae Corp. has provided clinical testing for more than 3.9 million patients¹, and is capable of estimating genetic prevalence in a database of 3.9M+ patient records. The data may be enriched for certain diseases, conditions, or otherwise. For example, the Invitae database is highly enriched for rare disease (FIGS. 3, 4A, and 4B). This type of enrichment, or for other reasons, may bias prevalence calculations. Therefore, to mitigate bias in the sample from which estimates of the prevalence of a genetic disorder are derived, only specific results, portions of data, or subsets of data may be used. Example volumes of filtered data include: SPINK1 (associated with hereditary pancreatitis): >1,240,000 and PRKN (associated with Parkinson's): >920,000. The Hardy-Weinberg equilibrium equation was used to derive prevalence estimates at the gene level for recessive, dominant, and X-linked conditions, using Invitae variants interpreted as pathogenic or likely pathogenic, in anonymized patients (see FIG. 5).

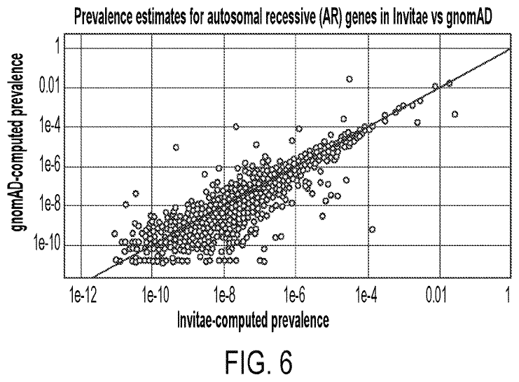

Prevalence estimates using Invitae data correlate with those derived from gnomAD (FIG. 6). Prevalence estimates were compared for autosomal recessive genes computed from gnomAD data vs those computed from Invitae data. Points on the graph represent individual genes, and the gray line represents the equality line, where gnomAD and Invitae estimates are equal (FIG. 6). There is a strong correlation between gnomAD and Invitae estimates (Spearman correlation=0.923)

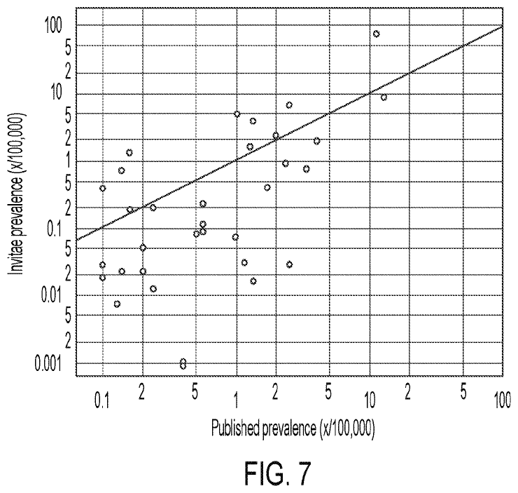

Invitae's genetic prevalence for autosomal recessive (AR) genes correlates with literature estimates. FIG. 7 demonstrates method performance on a set of genes with published prevalence.

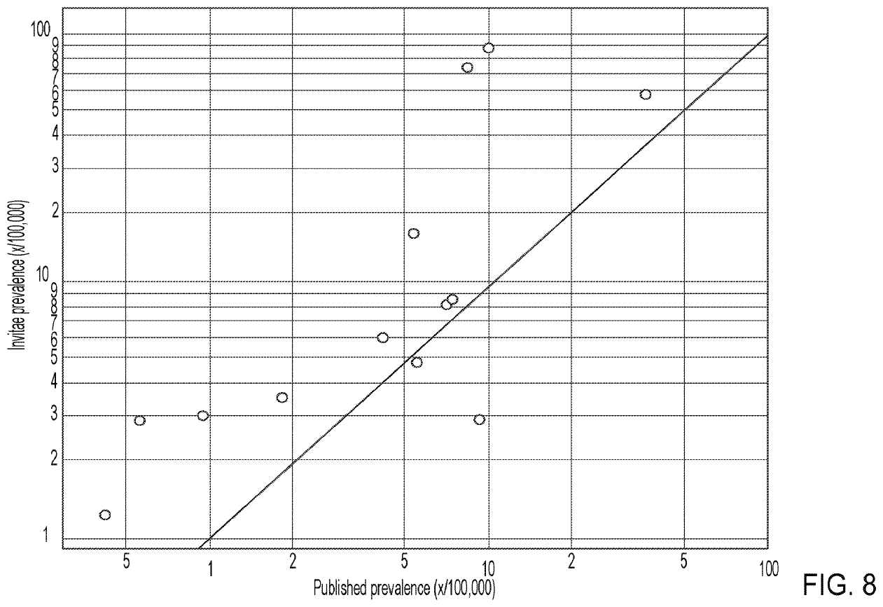

Next, it was determined whether prevalence estimates for autosomal recessive genes correlate with estimates from the literature. Prevalence estimates were gathered for dozens of genes from publication, typically epidemiology studies. The graph on FIG. 8 demonstrates that there is a good correlation between Invitae prevalence estimates and published prevalence: every point represents a single gene, and the gray line represents the equality line.

Invitae's genetic prevalence analysis for autosomal dominant (AD) genes correlates with literature estimates (FIG. 8). Studying the prevalence with Invitae data can be summarized as follows: Invitae prevalence estimates leverage a large population of tested patients, across clinical areas. After bias mitigation, estimates are consistent with literature reports. Due to the large volume of patients, large datasets, such as the Invitae dataset can be particularly useful to estimate prevalence for genes where pathogenic variants are very rare. To the best of inventor knowledge, the only other robust published method for large-scale autosomal dominant gene prevalence estimation, published by López-Rivera et al, presents three main short-comings: (1) The study only evaluates genes associated with neurodevelopmental disorders, or 3,207 genes, compared to the ~18,000 genes that can be estimated with the methods described herein; (2) this approach only applies to de novo mutations (as opposed to inherited); and (3) the method is not able to distinguish differences in prevalence between sex and ethnicity groups. Regarding (2) above, while many neurodevelopmental disorders are primarily caused by de novo mutations, the majority of disorders also have an inherited component. For many later-onset diseases, such as in cancer, most mutations are in fact inherited. Regarding (3) above, it is well known that inherited genetic mutations vary in prevalence between different populations. For example, the disorder autosomal dominant polycystic kidney disease (ADPKD), mentioned earlier, is primarily inherited. It is caused primarily by pathogenic variant in PKD1, PKD2, and neither genes have estimates published by López-Rivera et al.

Another related method, published by Gillentine et al., takes a similar approach to López-Rivera et al. (considering only de novo variants) and additionally only estimates prevalence within populations with neurodevelopmental disorders. doi:10.3390/biomedicines10112865.

In summary, our method is the only method that estimates prevalence of autosomal dominant gene variants taking into account both de novo and inherited variants for a general population across the whole exome, and breaks down prevalence by variants, sex, and ethnicity.

### REFERENCES

1. Invitae Reports First Quarter 2023 Financial Results. Invitae. May 9, 2023. Accessed May 30, 2023. ir.invitae.com/news-and-events/press-releases/press-release-details/2023/Invitae-Reports-First-Quarter-2023-Financial-Results/default.aspx.
2. Zhu W, Wang C, Mullapudi N, et al. A robust pipeline for ranking carrier frequencies of autosomal recessive and X-linked Mendelian disorders. NPJ Genom Med. 2022;7(1):72. doi:10.1038/s41525-022-00344-7
3. Johansen Taber K, Ben-Shachar R, Torres R, et al. A guidelines-consistent carrier screening panel that supports equity across diverse populations. Genet Med. 2022;24(1):201-213. doi:10.1016/j.gim.2021.09.009
4. López-Rivera J A, Pérez-Palma E, Symonds J, et al. A catalogue of new incidence estimates of monogenic neurodevelopmental disorders caused by de novo variants. Brain. 2020;143(4):1099-1105. doi:10.1093/brain/awaa051
5. Gillentine M A, Wang T, Eichler E E. Estimating the Prevalence of De Novo Monogenic Neurodevelopmental Disorders from Large Cohort Studies. Biomedicines. 2022;10(11):2865. Published 2022 Nov. 9

1. A method for estimating prevalence, in a population, of a mutation of a specific gene associated with a genetic disorder, the method comprising:
using at least one computer hardware processor to perform:
(a) obtaining a plurality of gene assay results for a corresponding plurality of subjects, the plurality of gene assay results comprising (i) gene panel results for a plurality of gene panels corresponding to a respective plurality of sets of requisitioned genes for the plurality of subjects, and (ii) background assay results corresponding to a plurality of non-requisitioned genes;
(b) selecting a subset of the plurality of subjects based on the plurality of sets of requisitioned genes and the specific gene associated with the genetic disorder, the selecting comprising, for each candidate subject:
determining, based on the plurality of sets of requisitioned genes and the specific gene, a measure of distance between the candidate subject and the specific gene; and
selecting the candidate subject to include in the subset of the plurality of subjects when the measure of distance is at least a threshold distance; and
(c) estimating prevalence of the mutation using assay results for the selected subset of the subjects.

2. The method of claim 1, wherein determining the measure of distance between the candidate subject and the specific gene comprises:
determining a distance between each set of requisitioned genes of the candidate subject and the specific gene to obtain one or more distances; and
determining the measure of distance between the candidate subject and the specific gene based on the one or more distances.

3. The method of claim 2, wherein determining the measure of distance between the candidate subject and the specific gene comprises determining a minimum distance of the one or more distances.

4. The method of claim 1, wherein determining a distance between the candidate subject and the specific gene comprises:
determining the distance as 0 when the plurality of requisitioned genes of the candidate subject comprises the specific gene; and
determining the distance as 1 when (a) the distance is not 0, and (b) the plurality of requisitioned genes of the candidate subject comprises a gene in common with a plurality of requisitioned genes of a second subject.

5. The method of claim 4, wherein determining a distance between the candidate subject and the specific gene comprises:
determining the distance as 2 when (a) the distance is not 0 or 1; and (b) the plurality of requisitioned genes of a second subject, the second subject having a distance 1 from the candidate subject, comprises a gene in common with a plurality of requisitioned genes of a third subject.

6. The method of claim 1, wherein the threshold distance is at least 1.

7. The method of claim 1, wherein the threshold distance is at least 2.

8. The method of claim 1, wherein estimating prevalence of the mutation comprises estimating prevalence of a mutation associated with a dominant genetic disorder.

9. The method of claim 1, wherein estimating prevalence of the mutation comprises estimating of de nova mutation associated with the genetic disorder.

10. The method of claim 1, wherein estimating prevalence of the mutation comprises estimating prevalence of a mutation associated with a recessive genetic disorder.

11. The method of claim 1, wherein estimating prevalence of the mutation comprises estimating prevalence of an inherited mutation associated with the genetic disorder.

12. The method of claim 1, wherein the plurality of gene panel results comprises at least 100,000 gene panel results.

13. The method of claim 1, wherein estimating prevalence of the mutation comprises estimating using Hardy-Weinberg equilibrium equation.

14. The method of claim 1, wherein estimating prevalence of the mutation comprises estimating in a population of a single sex.

15. (canceled)

16. The method of claim 1, further comprises estimating the prevalence of a plurality of mutations of a specific gene that are associated with the genetic disorder, the estimating comprising repeating steps (a)-(c) for each mutation of the plurality of mutations.

17. The method of claim 1, further comprising estimating the prevalence of the genetic disorder using the prevalence of the mutation or the plurality of mutations of claim 16.

18. The method of claim 16, further comprising identifying a mutation of the plurality of mutations that has a highest prevalence.

19.-22. (canceled)

23. A method of determining whether two mutations of a specific gene are likely to occur in cis or trans in a subject having or suspected of having a genetic disorder associated with mutation of the specific gene, the method comprising:
using at least one computer hardware processor to perform:
(a) obtaining a plurality of gene assay results for a corresponding plurality of subjects, each gene assay result comprising (i) gene panel results corresponding to a plurality of requisitioned genes and (ii) background results corresponding to a plurality of non-requisitioned genes;
(b) selecting a subset of the subjects based on the plurality of requisitioned genes and the specific gene, the selecting comprising, for each candidate subject: determining a measure of distance between the candidate subject and the specific gene; and selecting the candidate subject to include in the subset of subjects when the measure of distance is at least a threshold distance;
(c) identifying the two mutations of the specific gene in the gene assay results of the selected subset of subjects; and
(d) determining, using the two mutations identified in (c), whether the two mutations of a specific gene are likely to occur in cis or trans in the subject.

24. The method of claim 23, wherein determining whether two mutations of a specific gene are likely to occur in cis or trans in the subject comprises:
(i) determining, using the two mutations identified in (c), that the two mutations are likely to occur in cis in the subject when the two mutations are found in the same gene assay result of the gene assay results at least a threshold number of times; or
(ii) determining, using the two mutations identified in (c ), that the two mutations are likely to occur in trans in the subject when the two mutations are not found in the same gene assay result of the gene assay results at least a threshold number of times.

25. A method of determining whether a mutation of a given gene is associated with a genetic disorder, the method comprising:
using at least one computer hardware processor to perform:
(a) obtaining a plurality of gene assay results for a corresponding plurality of subjects, each gene assay result comprising (i) gene panel results corresponding to a plurality of requisitioned genes and (ii) background results corresponding to a plurality of non-requisitioned genes;
(b) selecting a first subset of the subjects based on the plurality of requisitioned genes and the given gene, the selecting comprising, for each candidate subject: determining a measure of distance between the candidate subject and the given gene; and selecting the candidate subject to include in the first subset of subjects when the measure of distance is at least a threshold distance;
(c) selecting a second subset of the subjects, the selecting comprising, for each of candidate subject:
selecting the candidate subject to include in the second subset of subjects when the candidate subject has, is suspected of having, or is being tested for the genetic disorder; and
(d) determining whether the mutation is associated with the genetic disorder using gene assay results of the first subset of subjects and gene assay results of the second subset of subjects.

26.-28. (canceled)
